(19) United States
(12) Patent Application Publication
(10) Pub. No.: US 2015/0080993 A1
(43) Pub. Date: Mar. 19, 2015

(54) CONTRACTIBLE LOW-FREQUENCY ELECTROTHERAPY APPARATUS

(71) Applicant: TRANSLAND Technology Co., Ltd., Taipei (TW)

(72) Inventor: Hung-Wen Wei, New Taipei City (TW)

(73) Assignee: TRANSLAND Technology Co., Ltd., Taipei (TW)

(21) Appl. No.: 14/031,395

(22) Filed: Sep. 19, 2013

(51) Int. Cl. A61N 1/04

(52) U.S. Cl. CPC A61N 1/0456
USPC 607/115

(57) ABSTRACT

A contractible low-frequency electrotherapy apparatus comprises a flexible carrying member and an electric stimulation member. The flexible carrying member includes a sleeving member having at least one flexible serpentine region with at least one extension unit. Each extension unit has two extension segments symmetric to each other and a bendable segment connected with the two extension segments and deformed by external force. The electric stimulation member includes a pulse generation unit, a pulse transmission conductor electrically connected with the pulse generation unit, and an electrode member electrically connected with the pulse transmission conductor. The pulse transmission conductor has at least one electric conduction unit embedded inside the extension unit of the flexible serpentine region. Each electric conduction unit has two connection segments corresponding to the extension segments and a reversal segment corresponding to the bendable segment.

### FIELD OF THE INVENTION

The present invention relates to an electrotherapy apparatus, particularly to a contractible low-frequency electrotherapy apparatus.

### BACKGROUND OF THE INVENTION

Long-term fatigue or excessive exercise usually causes muscle pains. Appropriate relaxation or massage of muscle or acupoint may relieve muscle pains. Further, modern medicine provides a TENS (Transcutaneous Electrical Nerve Stimulation) technology to solve the abovementioned problem, wherein an electric stimulator outputs a low-frequency pulse current to enhance blood circulation and relieve pain. Normally, TENS applies low-voltage electric pulses of different frequencies and amplitudes to the lesions or acupoints to relieve discomfort. Some researches show that medium-frequency (1000-100000 Hz) pulse current can also enhance blood circulation and relieve some special exercise-induced muscle pains.

The low-frequency electrotherapy apparatuses available in the market have only a single diameter to surround the limb of the user. In other words, each conventional low-frequency electrotherapy apparatus is designed only for the users of a specified body size. If the user cannot use one low-frequency electrotherapy apparatus, he has to try another one or adopt an auxiliary device to match his body size. It is very inconvenient for the user. Further, the user may have to buy different-sized electrotherapy apparatuses or auxiliary devices for his family members. In application of the low-frequency electrotherapy apparatuses available in the market, a conductive pad is attached to the lesion, and the electric pulse signal is transmitted to the conductive pad via a wire, whereby to apply electrotherapy to the user. However, the wire is likely to entangle the user and inconvenient to use. Besides, the wire has to connect with a host unit of the electrotherapy apparatus to obtain the electric pulse signal. During electrotherapy, the host unit is held by the user or placed in a certain position. Thus, the movement of the user is pretty restricted. Besides, the host unit is normally too bulky to conceal while the user carries about it. There have been electronic pain-relief patches available in the market. Although the electronic pain-relief patches are free of wires, they cannot be adjusted to adapt to the body size of the user or the location of the lesion. Therefore, the application thereof is limited.

### SUMMARY OF THE INVENTION

The primary objective of the present invention is to solve the problem that the conventional low-frequency electrotherapy apparatus cannot be adjusted to adapt to limbs of users.

To achieve the abovementioned objective, the present invention provides a contractible low-frequency electrotherapy apparatus, which comprises a flexible carrying member and an electric stimulation member. The flexible carrying member includes a carrying seat, and a sleeving member connected with the carrying seat and able to surround a limb of a user. The sleeving member has at least one flexible serpentine region formed on the sleeving member. Each flexible serpentine region includes at least one extension unit. Each extension unit has two extension segments symmetric to each other and a bendable segment connected with the two extension segments and deformed by external force. The electric stimulation member is arranged in the carrying seat and includes pulse generation unit triggered to generate an electrotherapy pulse signal, a pulse transmission conductor electrically connected with the pulse generation unit to transmit the electrotherapy pulse signal, and an electrode member electrically connected with the pulse transmission conductor to output the electrotherapy pulse signal to the limb of the user. The pulse transmission conductor has at least one electric conduction unit embedded inside the extension unit of the flexible serpentine region. Each electric conduction unit has two connection segments corresponding to the extension segments and a reversal segment corresponding to the bendable segment.

In one embodiment, the flexible carrying member further includes an attachment member protruding from a lateral of the sleeving member and revealing the electrode member.

In one embodiment, the extension plane of the attachment member intersects the extension plane of the sleeving member at a tilt angle.

In one embodiment, the flexible carrying member further includes an accommodation notch arranged at the attachment member to accommodate the electrode member.

In one embodiment, the flexible carrying member has a first flexible serpentine region and a second flexible serpentine region respectively arranged in laterals of the carrying seat; the length of the first flexible serpentine region is greater than that of the second flexible serpentine region.

In one embodiment, the flexible carrying member has a first flexible serpentine region and a second flexible serpentine region respectively arranged in laterals of the carrying seat; the length of the first flexible serpentine region is equal to that of the second flexible serpentine region.

In one embodiment, the sleeving member includes a first joining section and a second joining section that can be joined to the first joining section.

In one embodiment, the electric stimulation member further includes a power supply module and a circuit board connected with the power supply module and receiving power from the power supply module to generate the electrotherapy pulse signal.

In one embodiment, the electric stimulation member further includes a control switch connected with the circuit board to control the circuit board to output the electrotherapy pulse signal.

In one embodiment, the electric stimulation member has a conductive gel layer arranged on one side of the electrode member, which faces the limb of the user.

Compared with the conventional low-frequency electrotherapy apparatuses, the present invention is characterized in

1. The extension units and the electric conduction units in the flexible serpentine regions can be deformed, wherefore the flexible serpentine regions can be compressed or stretched to enable the present invention to surround fittingly the lesion of an arbitrary user.

2. The bendable segments of the flexible serpentine regions do not warp the skin of the user completely but only contact the lesion of the user, wherefore the skin of the user is exempted from irritation.

### BRIEF DESCRIPTION OF THE DRAWINGS

FIG. 1 is a top view schematically showing a contractible low-frequency electrotherapy apparatus according to one embodiment of the present invention;

FIG. 2A is a sectional view taken along Line 2A-2A in FIG. 1 and schematically showing a contractible low-frequency electrotherapy apparatus according to one embodiment of the present invention;

FIG. 2B is a locally-enlarged view of FIG. 2A and schematically showing a contractible low-frequency electrotherapy apparatus according to one embodiment of the present invention;

FIG. 3 is a sectional view taken along Line 3-3 in FIG. 1 and schematically showing a contractible low-frequency electrotherapy apparatus according to one embodiment of the present invention;

FIG. 4 is a perspective view schematically showing the appearance of a contractible low-frequency electrotherapy apparatus according to one embodiment of the present invention; and

FIG. 5 is a perspective view schematically showing the application of a contractible low-frequency electrotherapy apparatus according to one embodiment of the present invention.

### DETAILED DESCRIPTION OF THE PREFERRED EMBODIMENTS

The technical contents of the present invention are described in detail in cooperation with the drawings below.

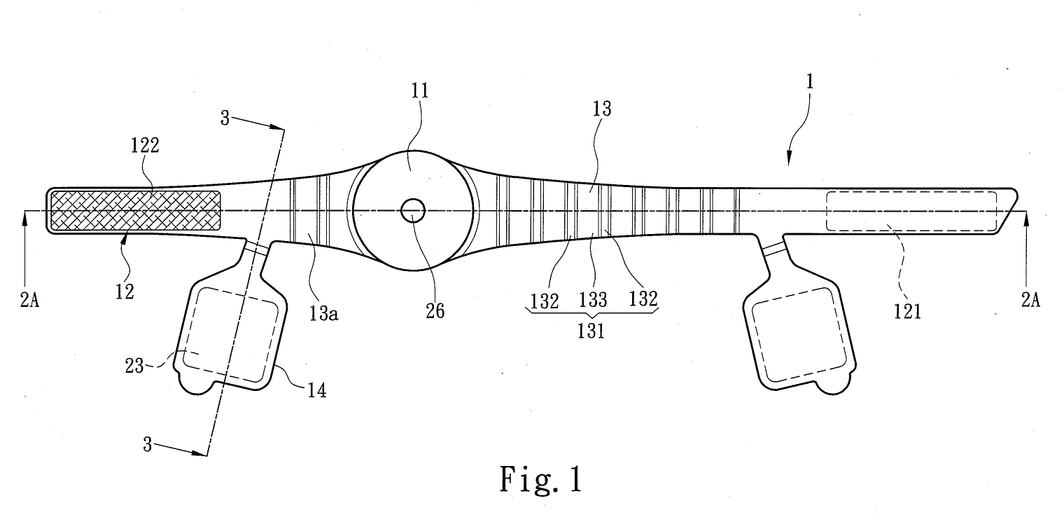

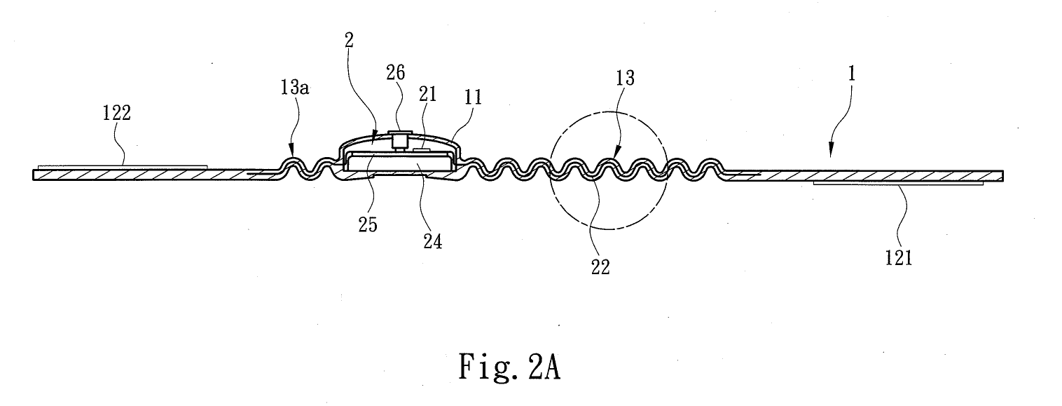

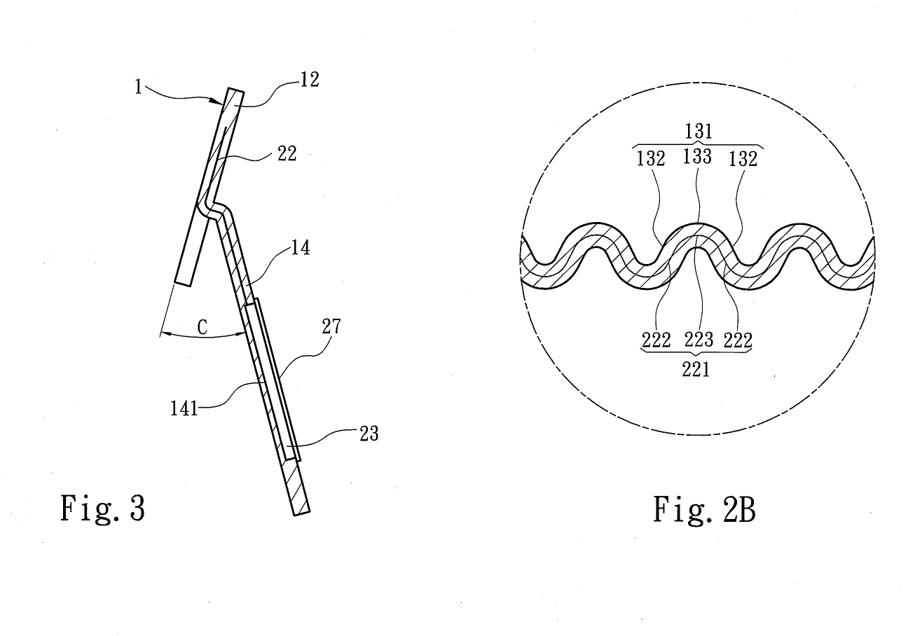

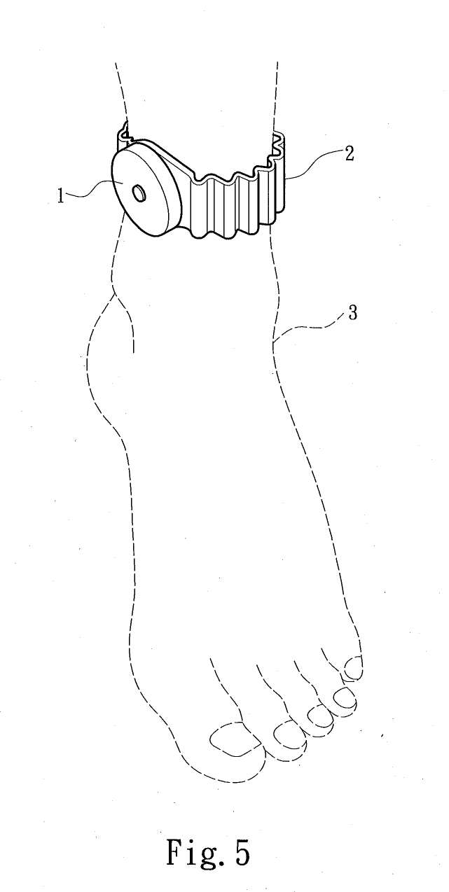

Refer to FIGS. 1-5. The contractible low-frequency electrotherapy apparatus of the present invention can annularly surround a limb 3 of a user and outputs an electrotherapy pulse signal to heal the limb 3 of the user. The contractible low-frequency electrotherapy apparatus of the present invention comprises a flexible carrying member 1 and an electric stimulation member 2. The flexible carrying member 1 includes a carrying seat 11, a sleeving member 12 connected with the carrying seat 11 and able to surround a limb of a user, and at least one flexible serpentine region formed on the sleeving member 12. Each flexible serpentine region has at least one extension unit 131. Each extension unit 131 has two extension segments 132 symmetric to each other and a bendable segment 133 connected with the two extension segments 132 and deformed by external force. In one embodiment, the flexible carrying member 1 is a one-piece component fabricated via inject-molding or hot-pressing a flexible material (such as a rubber material or a silicone material). The sleeving member 12 includes a first joining section 121 and a second joining section 122 that can be joined to the first joining section 121. In one embodiment, the first joining section 121 and the second joining section 122 are hook-and-loop fasteners or other fasteners. The first joining section 121 and the second joining section 122 are joined to each other to make the flexible carrying member 1 form an annular shape sleeving a limb of a user. The users have different body sizes: some are slender, and some are stout. The flexible serpentine regions are designed to be applicable to users of different body sizes. In the case that the user is fleshy, the extension units 131 of the flexible serpentine regions can be extended to make the flexible carrying member 1 have a diameter about equal to the diameter of the limb of the user, whereby the flexible carrying member 1 can securely surround the limb of the user. In the embodiment shown in FIG. 2, the flexible carrying member 1 has a plurality of flexible serpentine regions, including a first flexible serpentine region 13 and a second flexible serpentine region 13a, wherein the length of the first flexible serpentine region 13 is greater than or equal to that of the second flexible serpentine region 13a.

In the present invention, the electric stimulation member 2 is arranged in the carrying seat 11 and includes a pulse generation unit 21 triggered to generate the electrotherapy pulse signal, a pulse transmission conductor 22 electrically connected with the pulse generation unit 21 to transmit the electrotherapy pulse signal, and an electrode member 23 electrically connected with the pulse transmission conductor 22 to output the electrotherapy pulse signal to a limb of a user. The pulse transmission conductor 22 has at least one electric conduction unit 221 embedded inside the extension unit 131 of the flexible serpentine region, such as the first flexible serpentine region 13 or the second flexible serpentine region 13a. Each electric conduction unit 221 has two connection segments 222 corresponding to the extension segments 132 and a reversal segment 223 corresponding to the bendable segment 133. In one embodiment, the electric stimulation member 2 further includes a power supply module 24, a circuit board 25 connected with the power supply module 24 and receiving power from the power supply module 24 to generate the electrotherapy pulse signal, and a control switch 26 connected with the circuit board 25 to control the circuit board 25 to output the electrotherapy pulse signal. The electric conduction unit 221 is fabricated to have a shape identical to the shape of the extension unit 131. For example, if the extension unit 131 has a wave shape, the electric conduction unit 221 is also fabricated to have the wave shape. While the flexible serpentine region, such as the first flexible serpentine region 13 or the second flexible serpentine region 13a, is stretched to deform by external force, the electric conduction unit 221 is also stretched and deformed. Therefore, via adjusting the flexible carrying member 1, the contractible low-frequency electrotherapy apparatus of the present invention can be adjusted to adapt to the body size of the user and accurately correspond to a lesion of the limb of the user. In one embodiment, the electric stimulation member 2 has a conductive gel layer 27 arranged on one side of the electrode member 23, which faces the limb of the user.

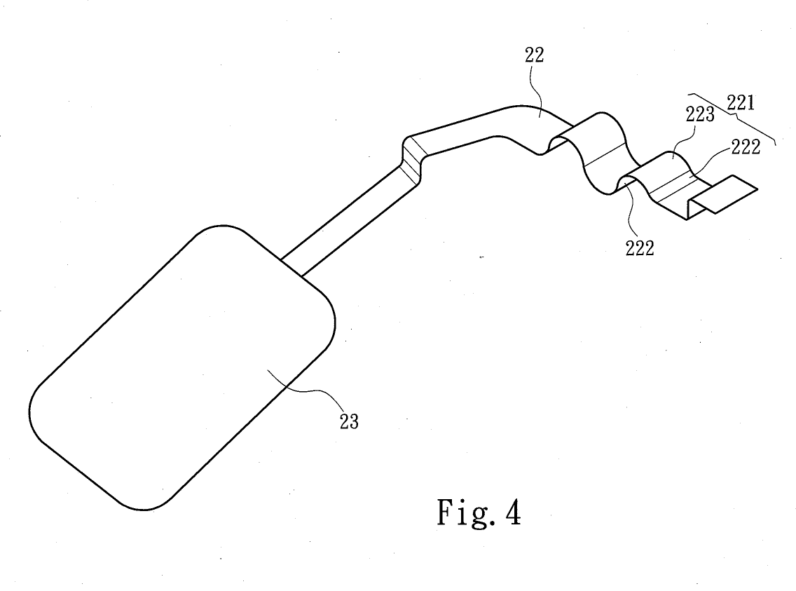

Refer to FIG. 1 and FIG. 4. In one embodiment, the flexible carrying member 1 further includes an attachment member 14 protruding from the lateral of the sleeving member 12 and revealing the electrode member 23. The extension plane of the attachment member 14 intersects the extension plane of the sleeving member 12 at a tilt angle of C degrees, whereby the attachment member 14 can attach to the limb of the user more ergonomically. In one embodiment, the flexible carrying member 1 further includes an accommodation notch 141 arranged at the attachment member 14 to accommodate the electrode member 23. The shape of the accommodation notch 141 is varied with the shape of the electrode member 23.

In conclusion, the present invention proposes a contractible low-frequency electrotherapy apparatus, which comprises a flexible carrying member and an electric stimulation member. The flexible carrying member includes a sleeving member. The sleeving member has at least one flexible serpentine region containing at least one extension unit. Each extension unit has two extension segments symmetric to each other and a bendable segment connected with the two extension segments and deformed by external force. The electric stimulation member includes a pulse generation unit, a pulse transmission conductor electrically connected with the pulse generation unit, and an electrode member electrically connected with the pulse transmission conductor. The pulse transmission conductor has at least one electric conduction unit embedded inside the extension unit. Each electric conduction unit has two connection segments corresponding to the extension segments and a reversal segment corresponding to the bendable segment. As the flexible serpentine region enables the sleeving member to stretch appropriately, the contractible low-frequency electrotherapy apparatus of the present invention can be adjusted to adapt to the body sizes of different users.

What is claimed is:

1. A contractible low-frequency electrotherapy apparatus comprising
a flexible carrying member including a carrying seat, and a sleeving member connected with the carrying seat and able to surround a limb of a user, the sleeving member having at least one flexible serpentine region, each flexible serpentine region including at least one extension unit, wherein each extension unit has two extension segments symmetric to each other and a bendable segment connected with the two extension segments and deformed by external force; and
an electric stimulation member arranged in the carrying seat and including a pulse generation unit triggered to generate an electrotherapy pulse signal, a pulse transmission conductor electrically connected with the pulse generation unit to transmit the electrotherapy pulse signal, and an electrode member electrically connected with the pulse transmission conductor to output the electrotherapy pulse signal to the limb of the user, wherein the pulse transmission conductor has at least one electric conduction unit embedded inside the extension unit of the flexible serpentine region, and wherein each electric conduction unit has two connection segments corresponding to the extension segments and a reversal segment corresponding to the bendable segment.

2. The contractible low-frequency electrotherapy apparatus according to claim 1, wherein the flexible carrying member includes an attachment member protruding from a lateral of the sleeving member and revealing the electrode member.

3. The contractible low-frequency electrotherapy apparatus according to claim 2, wherein an extension plane of the attachment member intersects an extension plane of the sleeving member at a tilt angle.

4. The contractible low-frequency electrotherapy apparatus according to claim 2, wherein the flexible carrying member includes an accommodation notch arranged at the attachment member to accommodate the electrode member.

5. The contractible low-frequency electrotherapy apparatus according to claim 1, wherein the flexible carrying member has a first flexible serpentine region and a second flexible serpentine region respectively arranged in laterals of the carrying seat, and wherein a length of the first flexible serpentine region is greater than that of the second flexible serpentine region.

6. The contractible low-frequency electrotherapy apparatus according to claim 1, wherein the flexible carrying member has a first flexible serpentine region and a second flexible serpentine region respectively arranged in laterals of the carrying seat, and wherein a length of the first flexible serpentine region is equal to that of the second flexible serpentine region.

7. The contractible low-frequency electrotherapy apparatus according to claim 1, wherein the sleeving member includes a first joining section and a second joining section, that can be joined to the first joining section.

8. The contractible low-frequency electrotherapy apparatus according to claim 1, wherein the electric stimulation member includes a power supply module and a circuit board connected with the power supply module and receiving power from the power supply module to generate the electrotherapy pulse signal.

9. The contractible low-frequency electrotherapy apparatus according to claim 8, wherein the electric stimulation member includes a control switch connected with the circuit board to control the circuit board to output the electrotherapy pulse signal.

10. The contractible low-frequency electrotherapy apparatus according to claim 1, wherein the electric stimulation member has a conductive gel layer arranged on one side of the electrode member.
